(19) United States
(12) Patent Application Publication
(10) Pub. No.: US 2014/0051832 A1
(43) Pub. Date: Feb. 20, 2014

(54) METHOD FOR THE PRODUCTION OF RECOMBINANT HUMAN FACTOR VIII

(71) Applicants: Marcos A. A. Demasi, São Paulo (BR); Mari Cleide Sogayar, São Paulo (BR); Christian Colin, São Paulo (BR)

(72) Inventors: Marcos A. A. Demasi, São Paulo (BR); Mari Cleide Sogayar, São Paulo (BR); Christian Colin, São Paulo (BR)

(73) Assignee: UNIVERSIDADE DE SÃO PAULO - USP, São Paulo (BR)

(21) Appl. No.: 14/004,235

(22) PCT Filed: Mar. 11, 2011
(86) PCT No.: PCT/BR11/00069

§ 371 Date: Nov. 6, 2013

(51) Int. Cl. C07K 14/755

(52) U.S. Cl. CPC C07K 14/755
USPC 530/383; 435/69.6

(57) ABSTRACT

The object of the present invention is to provide methods for the production of recombinant human Factor VIII, employing specific endoproteases, thus assuring full proteolytic processing of said factor even during its biosynthesis, consequently avoiding additional purification steps. Other objects of the present invention are the recombinant human Factor VIII as obtained by said methods, pharmaceutical compositions, related uses and therapeutic methods.

### FIELD OF THE INVENTION

The object of the present invention is to provide methods for the production of recombinant human Factor VIII, employing specific endoproteases, thus assuring full proteolytic processing of said factor even during or after its biosynthesis, consequently avoiding additional purification steps. Other objects of the present invention are the recombinant human Factor VIII as obtained by said methods, pharmaceutical compositions, related uses and therapeutic methods.

### BACKGROUND OF THE INVENTION

Hemophilia is a genetic dysfunction disabling the body to control bleedings (hemorrhagic diathesis), due to the reduction, inactivation or lack of one of the coagulation factors as required to form a blood clot.

Excessive bleeding may be external, if the skin is damaged by a cut or abrasion, or internal, on muscles, articulations or organs. Hematomas, hemarthrosis (bleedings on articulations) and intracranial bleedings frequently appear and, in the lack of an effective treatment, may even cause death.

The dysfunction may be classified into three types:
Hemophilia A—characterized by the lack of the coagulation Factor VIII (FVIII), or anti-hemophilic globulin.
Hemophilia B—or Christmas disease, characterized by the lack of the hemophilic factor B or factor IX.
Hemophilia C—determined by a dominant autosomic gene not related to gender, characterized by the lack of a factor called PTA or factor XI.

Hemophilia A, characterized by the lack, inactivation or partial activation of the coagulation Factor VIII, mainly affects male patients, being the most common kind against diagnosed patients.

The reduction of Factor VIII is also related to von Willebrand's disease, which is another hereditary dysfunction characterized by abnormal slow speed of blood coagulation, causing spontaneous and long nose and gum bleedings, affecting both men and women. Von Willebrand's factor is responsible for the protection and stabilization of Factor VIII and, in its absence, there is a consequent reduction of Factor VIII.

Human Factor VIII, which is essential for blood coagulation, is a non-enzymatic glycoprotein present in blood plasma, acting in the intrinsic route of the blood coagulation cascade, as a co-factor of serine-protease factor IX, in the step of proteolytic activation of factor X.

Patients with diseases related to the reduction or lack of Factor VIII, such as hemophilia A, must increase the levels of Factor VIII in the blood by means of administration of exogenous Factor VIII, thus avoiding hemorrhagic occurrences and expontaneous bleeding.

This is provided by a “factor reposition therapy”, usually made intravenously. Such treatment started in the 1950s, with direct plasma transfusion, and later with the use of Factor VIII concentrates, both obtained from plasma from blood donators.

During the 1970s and 1980s, the lack of other routes of treatment and the lack of technology to diagnose a few viruses, such as the Acquired Immunodeficiency Syndrome (AIDS) virus, caused a large scale contamination in the hemophilic population.

Initially, concentrates obtained from human Factor VIII also included contaminants, but technical advancements, added to the adoption of a range of steps concerning the method to obtain them, made their use become safer. However, besides the theoretical risk still existent towards the eventual appearance of a pathogen (e. g. a still not characterized virus, resistant against the viral inactivation and purification methods as used), there are still problems concerning the reduced quantity of donators and the very low concentrations of Factor VIII as present in the plasma.

These facts make the method to obtain highly purified Factor VIII preparations from donators' plasma become very complex and expensive, not only due to the need of large quantities of human plasma, a rarer and rarer material, but also for the complexity in terms of quality control of the final product to be given to patients with dysfunctions related to the reduction, inactivation or lack of coagulation Factor VIII.

Since the 1990s, an alternative form of production of human Factor VIII has been adopted, involving the stable transfection of the nucleotide sequence corresponding to the protein-coding region of the human Factor VIII gene into the genome of animal cells in culture. Therefore, said cells start stably producing human Factor VIII in culture and to secrete Factor VIII in the cultivation medium, from which it may be purified later on.

Human Factor VIII as produced in this way is known as recombinant human Factor VIII. In comparison with Factor VIII concentrates as obtained from plasma from human donators, recombinant human Factor VIII is considered as a potentially non-exhaustive and safer source.

To obtain cells in culture producing recombinant human Factor VIII, both the full coding region of the Factor VIII gene or portions of the coding region which are enough to obtain a functional product have been used.

The second-generation recombinant product, as commercialized by the Wyeth group under the commercial name Re-Facto, is produced by using two portions of the full coding region of human Factor VIII connected by an artificial connection sequence. On the other hand, first-generation recombinant products, commercialized by the Bayer group under the commercial names Kogenate, Recombinate or Helixate, are produced by using the full coding region of the human Factor VIII gene.

The production of recombinant human Factor VIII from its full coding region presents a range of obstacles, taking the expression in an industrial platform to unsatisfactory levels.

One of the alternatives used to overcome such problem has been the use of portions of the coding region of Factor VIII which are enough to obtain a functional product. The most explored changes are the partial or full deletion of a portion of the coding region for Factor VIII, coding the central part of the molecule, known as domain B. Said changes allow for higher production of Factor VIII, although also industrially unsatisfactory (Saenko, E. L., Ananyeva, N. M., Shima, M., Hauser, C. A. & Pipe, S. W. (2003). The future of recombinant coagulation factors. J Thromb Haemost, 1, 922-30).

In the full deletion of the portion of the coding region for Factor VIII corresponding to domain B, coding regions for the remaining portions of the molecule, known as heavy and light chains, are connected by using an artificial connection sequence, as used in the commercial product Re-Facto as mentioned above. Said process also presents low industrial yields (Lind P., Larsson K., Spira J., Sydow-Backman M., Almstedt A., Gray E. and Sandberg H. (1995). Novel forms of B-domain-deleted recombinant Factor VIII molecules construction and biochemical characterization. Eur. J. Biochem. 232, 19-27).

A second alternative involves partial deletions of the portion of the coding region for Factor VIII corresponding to domain B and the separate production of heavy and light chains of Factor VIII. In this configuration, excerpts of variable extensions, corresponding to domain B, are connected to the coding region for light chain (U.S. Pat. No. 5,693,499, by Chemo Sero Therapeut Res Inst (JP)).

Both in the full deletion and in the partial deletion as disclosed above, synthesized products must be proteolytically processed. In the case of full deletion, proteolytic processing is required for the physical separation between heavy and light chains and the formation of active Factor VIII to occur. In the case of partial deletion, however, the proteolytic processing of the light chain fused to the excerpt of domain B must occur, so that the light chain as produced is identical to the one as naturally found in human plasma.

Proteolytic processing also occurs naturally, even when the full coding portion is used, i. e. mammal cells as used to produce Factor VIII have the ability to proteolytically process Factor VIII, despite not knowing which enzyme is responsible for said activity. However, this capacity is very limited, i. e. when said cells produce higher quantities of Factor VIII, said proteolytic processing is always partial.

No matter which is the used alternative, the main problem is still the partial or incomplete proteolytic processing of the products as formed. This causes the production of artificial forms of Factor VIII (Factor VIII complex) which, if present in the final pharmaceutical product, could present unexpected antigenic properties to the patient to be treated.

Furthermore, said incomplete processing reduces the quantity of the final product as obtained, considering that part of what is being produced by the animal cells is being converted into a subproduct, i. e. into artificial forms of Factor VIII which need to be excluded in some part of the production method.

This not only results in less yielding of the final product, but also in a longer and more expensive method.

Causes of such an incomplete proteolytic processing have not been fully clarified. A few hypotheses would be: a) saturation of protease(s) as naturally present in cells used to produce large quantities of Factor VIII; or b) eventual differences in activity and specificity between protease(s) as present in non-human cells used to produce Factor VIII and human protease(s) naturally performing said processing.

According to the hypothesis b) above, although cells used to produce Factor VIII are able to proteolytically process said factor, they do so only partially, since said cells do not have protease(s) naturally performing said processing.

Therefore, considering the possibility of partial or incomplete proteolytic processing, the process to produce recombinant Factor VIII will be longer, since it always requires a purification step for control of eventual forms derived from Factor VIII.

The technology proposal for full proteolytic processing has been little explored, since there is full specificity between endoprotease and precursor, making it difficult and costly to search which endoprotease is able to process a given precursor. Furthermore, theoretical methods are not efficient to indicate possibly effective endoprotease(s). This means that, despite a given protein theoretically has a site recognized by endoproteases, it is not possible to realize which one would be able to effectively recognize said site.

The state of the art teaches the use of different endoproteses in the proteolytic processing of different blood coagulation factors, confirming the specificity between endoprotease and precursor, but lacking to propose an effective means to guarantee the full proteolytic processing of human coagulation Factor VIII, as previously explained.

Patent U.S. Pat. No. 5,965,425 by Genetics Inst (US), published on Oct. 12, 1999, teaches the use of specific endoprotease in a method to increase the efficiency of proteolytic processing of precursor polypeptides for factor VII, factor IX, factor X, protein C, protein S, prothrombin and von Willebrand's factor, in recombinant host cells, aiming to guarantee a correct and efficient fold, thus providing increase in activity.

The international patent application WO 03/100053, by the Philadelphia Children Hospital, published on Dec. 4, 2003, teaches the use of an artificial site recognized by a specific endoprotease. More specifically, variants as disclosed have full deletion of the DNA sequence corresponding to the domain B and use the sequences corresponding to heavy and light chains, or their portions, interconnected by a coding DNA segment for an artificial cleaving site of the specific endoprotease. Concerning the heavy chain, the coding region of the natural heavy chain (amino acids 1-470) or their variants are used, causing small deletions of the C-terminal end of heavy chain (amino acids 1-700, 1-710, 1-720 and 1-730). Concerning the light chain, variants of the coding region for the light chain containing small deletions corresponding to the N-terminal end of the light chain are used. Most of these variants correspond to the amino acid sequence 1690-2232 and the other variants are little smaller portions than this sequence (amino acids 1700-2232, 1710-2232, 1720-2232 and 1730-2232).

The international patent application WO 2008/022151, by Inspiration Biopharmaceuticals (US), published on Feb. 21, 2008, discloses methods to produce factor IX by using the specific endoprotease PACE and enzymes VKOR and VKGC as required for the carboxylation of factor IX depending on vitamin K.

Therefore, there is still the need of enhancements to the production process for recombinant human Factor VIII, so to guarantee the full proteolytic processing, thus avoiding additional purification steps.

### BRIEF DESCRIPTION OF THE DRAWINGS

FIGS. 1A, 1B and 1C refer to configurations of the coding region for Factor VIII as used for its production.

FIG. 2 schematically and simply shows a process to produce separate heavy and light chains to obtain recombinant Factor VIII, wherein (*) shows the Factor VIII complex containing the light chain fused to the path of domain B due to incomplete proteolytic processing.

FIG. 3 schematically shows a part of the process to produce heavy and light chains to obtain Factor VIII, wherein B′ highlights the step guaranteeing full proteolytic processing in an embodiment of the present invention.

FIG. 4 schematically shows part of the process to produce heavy and light chains to obtain Factor VIII, wherein C′ highlights the step guaranteeing full proteolytic processing in an embodiment of the present invention and (*) shows the Factor VIII complex containing the light chain fused to the path of the domain B due to incomplete proteolytic processing.

FIGS. 5 and 6 show a representative result corresponding to the assays as made to evaluate the alternative for the simultaneous production of Factor VIII and endoproteases, in an embodiment of the present invention.

FIGS. 7 and 8 show a representative result corresponding to the assays made to evaluate the alternative to produce proteases in other cells and add them to the growing medium of cells producing Factor VIII.

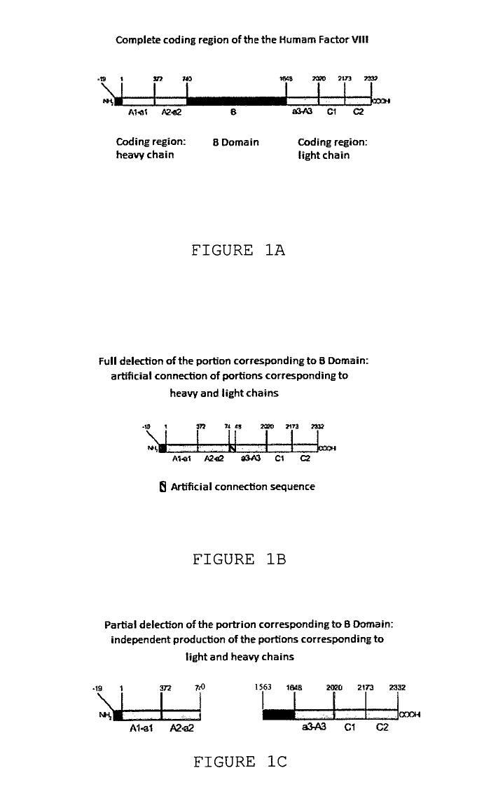

### DISCLOSURE OF THE INVENTION The Applicant has verified that three specific endoproteases are able to recognize natural sites existent in the link between domains B and the light chain.

Therefore, it has been possible to develop enhanced processes to produce recombinant human coagulation Factor VIII.

Therefore, the present invention refers to the use of specific enzymes, notably endoprotease PCSK5—Protein Convertase Subtilisin/Kexin type 5, also known as SPC6, and/or the isoform and endoprotease PCSK6—Protein Convertase Subtilisin/Kexin type 6, also known as PACE4—Paired basic Amino acid Cleaving Enzyme 4 and/or secreted mutant from the endoprotease PACE—Paired basic Amino acid Cleaving Enzyme, known as PACESOL, so to promote a full proteolytic processing of Factor VIII, still during the step of its production by animal cells, thus avoiding additional steps of purification and providing for enhanced methods to produce recombinant human coagulation Factor VIII.

Endoproteases PACE, SPC6 and PACE4 belong to a family of proteins acting in the processing of cell proteins which are initially synthesized as latent precursors (inactive forms), but need a portion of the molecule to be proteolytically removed to cause the production of its mature (active) form. Until now, only a few proteins which need SPC6 to be proteolytically processed are known. These proteins are subunits alpha of some proteins known as integrins. SPC6 seems to be also able to process proteins pro-renin, pro-matrix metalloprotease type 1 and glycoprotein gp160 of the HIV virus. Concerning PACE4, proteins of the growth transforming factor family, known as TGFpβ—Transforming Growth Factor β, pro-albumin and Von Willebrand's factor are examples of proteins which are proteolytically processed by said endoprotease. At least eight alternative transcripts linked to the human PACE4 gene are known, which code different isoforms designated as isoforms a to h.

PACESOL is an artificial mutant form of the protein PACE which does not have the residues 716-794. The

PACE protein is a member of the enzyme family able to Clive after paired basic amino acid residues.

Patent number U.S. Pat. No. 5,965,425 as mentioned above discloses the use of the endoprotease PACE and secondarily its variants, like the secreted mutant PACESOL, to promote the process of precursor proteins for some components of the coagulation cascade, especifically factor IX, protein C, protein S, prothrombin, factor X, factor VII and von Willebrand's factor, aiming to obtain them with an appropriate fold in its biologically active or mature forms. Coagulation Factor VIII has, in its polypeptidic sequence, the sequence of residues of amino acid Arg-Hys-Gln-Arg, which would theoretically be recognized by endoprotease PACE. Therefore, in a first moment, the extension of use of PACE for the promotion of proteolytic processing of Factor VIII could be expected. However, endoprotease components of the family of proteins of which PACE is a part present a few overlaying examples for given substrates, such as in the case of von Willebrand's factor, which is cleaved by both PACE and PACE4. In other cases, there is some specificity e.g. for factor IX, which apparently is only cleaved by PACE. However, in opposition to the art, the Applicant concluded that the wild PACE enzyme does not promote the cleavage of the junction between domain B and the light chain, as could be expected by analogy, disregarding the use of PACE and its variants in a first moment and confirming the knowledge that it is not usual to anticipate by which endoprotease(s) a given precursor may be processed.

Surprisingly, the Applicant verified that specific endoproteases SPC6, isoform a of PACE4 (PACE 4 AI) and PACESOL are able to promote the proteolytic process of recombinant human Factor VIII. Therefore, from this knowledge, it was possible to enhance methods to produce recombinant human Factor VIII, thus guaranteeing full proteolytic processing and avoiding an additional step of purification of Factor VIII complexes as eventually formed.

According to the present invention, “full proteolytic processing” is the non-formation of a substantial quantity of recombinant human coagulation

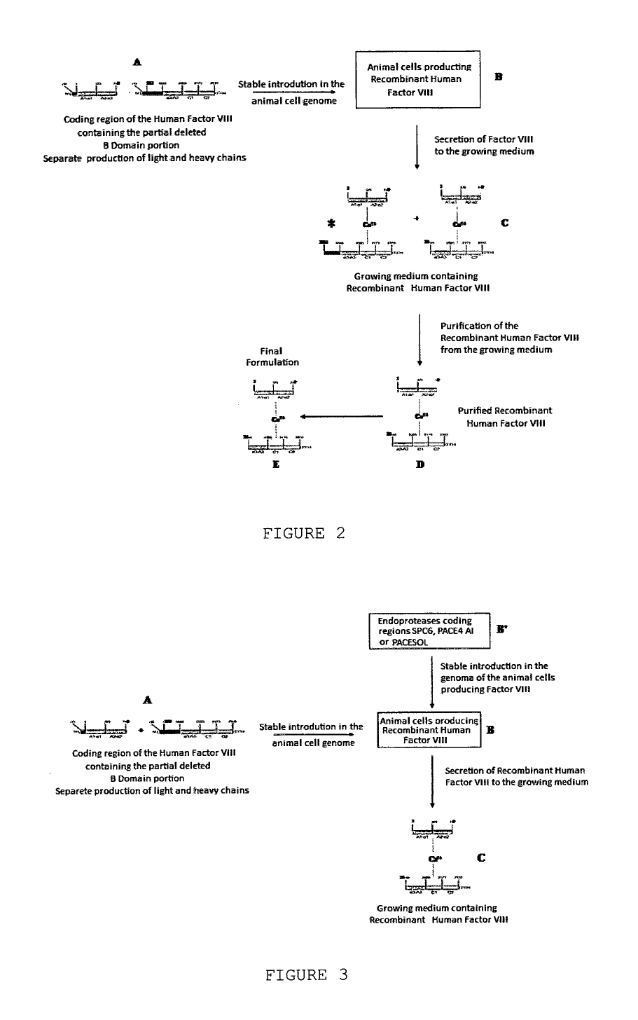

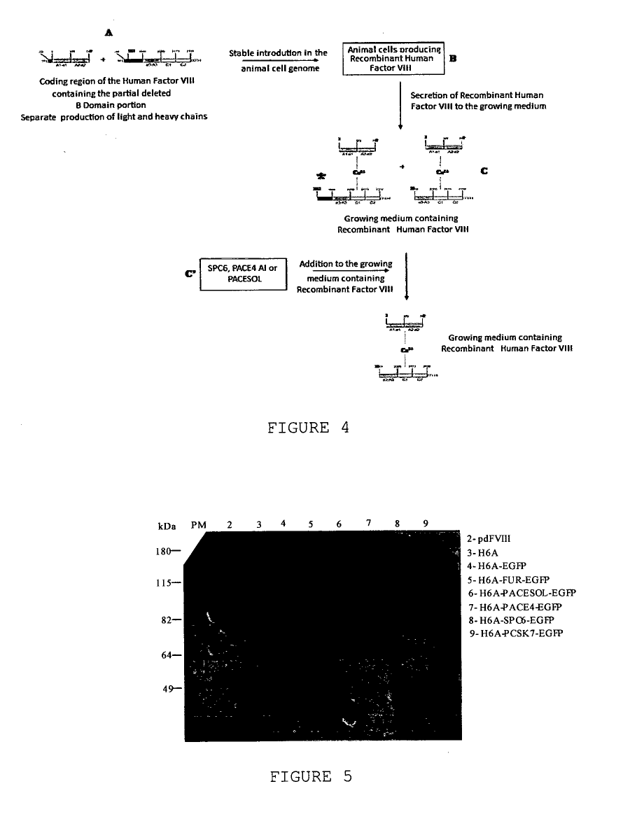

Factor VIII complexes containing the light chain fused to the path of domain B, as shown by FIGS. 2 and 4, thus avoiding additional purification steps.

By “substantial”, we understand a quantity which is not liable to cause damage to the patient, allowing eliminating an additional purification step.

The method of the present invention involves partial deletions of the portion of the coding region for Factor VIII corresponding to domain B and the independent production of heavy and light chains, as disclosed by the patent U.S. Pat. No. 5,693,499, incorporated herein as a reference. In this configuration, excerpts of variable extensions, corresponding to domain B, are connected to the coding region for the light chain.

The coding sequence for the light chain as used in the present invention corresponds to the natural non-processed amino acid sequence of the light chain (amino acids 1648-2232) linked to a path corresponding to a portion of domain B (amino acids 1563-1649). Said sequence has a natural site, adjacent to the amino acid 1648, recognized by pro-convertase type endoprotease(s), i. e. one or more members of the furin family.

The method of the present invention consists in (a) introduction of coding regions for endoproteases SPC6 and/or PACE4 AI and/or PACESOL in the genome of animal cells producing Factor VIII and/or (b) addition of endoproteases SPC6 and/or PACE4 AI and/or PACESOL to the growing medium containing recombinant Factor VIII.

In the method of the present invention, the portions of coding regions corresponding to the heavy chain of human Factor VIII and the light chain containing a small path corresponding to domain B of the human Factor VIII are stably inserted in the genome of growing animal cells, so that these cells start co-producing heavy and light chains of the human Factor VIII and therefore large quantities of the complex between heavy and light chains, with coagulant activity (Factor VIII).

Besides the above elements, in a first embodiment of the present invention, as shown by FIG. 3, coding regions for endoproteases PCSK5 (SPC6) and/or the isoform a of PCSK6 (PACE4 AI) and/or PACESOL are stably inserted in the genome of growing animal cells producing the recombinant human Factor VIII, so that such cells start to produce recombinant Factor VIII identically to what is naturally found in human plasma and free from artificial forms of Factor VIII (step B′).

In a second embodiment of the present invention, as shown by FIG. 4, coding regions for endoproteases SPC6 and/or PACE4 AI and/or PACESOL are stably inserted in the genome of growing animal cells, so that such cells may start to produce endoproteases PCSK5, PACE4 AI or PACESOL, to be used, jointly or separately, in the proteolytic processing of artificial forms of Factor VIII, by the addition of endoproteases to the growing medium containing cells producing recombinant human Factor VIII (step C′).

According to the present invention, the production of endoproteases SPC6, PACE4 AI or PACESOL to be used for the promotion of the proteolytic processing of

Factor VIII can be made in a wide variety of growing cells derived from animals, insects, fungi/yeasts and bacteriae as known in the art.

Alternatively, the production of human endoproteases SPC6, PACE4 AI and PACESOL may be effected by means of a cell-free system, e. g. by translating its coding region in vitro, by means of chemical synthesis of its purification from naturally producing cells.

In another embodiment, endoproteases SPC6, PACE4 AI and PACESOL may be produced so to have, in their sequences, non-natural amino acids or amino acid analogues, as long as keeping their endoproteolytic activities. In another embodiment of the present invention, steps B′ and C′ may be additively performed.

As a result, the present invention provides an enhanced method wherein there is the production of Factor VIII free from artificial forms presenting unpredictable and more effective antigenic potential, by the conversion of an eventually undesired subproduct, which should be eliminated in some step of the production method, into a Factor VIII which is identical to the one as naturally found in human plasma. According to the present invention, full coding regions of human endoproteases SPC6, PACE4 AI and PACESOL may be used or, alternatively, other configurations of the coding regions for endoproteases SPC6, PACE4 AI and PACESOL endoproteases, be them natural or artificial, may be used. In this embodiment, coding regions for SPC6, PACE4 AI and PACESOL which are different from the coding region concerning natural allelic variations or variants from other species may be used, as well as natural alternative forms of SPC6, PACE4 AI and PACESOL having the same proteolytic activity, such as isoforms caused by an alternative transcription of said genes and various post-translational processes, such as proteolytic processing.

In this sense, coding regions for endoproteases SPC6, PACE4 AI and PACESOL from other animal species, be them mutant, natural or artificial, may be used, as well as excerpts from the coding region for these genes, with ability to generate truncated polypeptides, fused or not to other polypeptides, but keeping the same activity of original proteins.

According to the present invention, as the promoting region for gene transcription, in case of production of light and heavy chains of Factor VIII, the hybrid promoter partly derived from the β-actin gene of chickens and also from the transcription enhancer derived from human cytomegalovirus is used. In case of the production of endoproteases SPC6, PACE 4 AI and PACESOL, the promoter derived from human cytomegalovirus is used.

Alternatively, with no limitation whatsoever, any strong promoting region, natural or artificial, which is compatible with the super production of the recombinant protein of interest, may be used. Examples of other strong natural promoters which may be used include initial and late promoting regions derived from the SV40 virus, the promoting region derived from the late region of adenovirus and the Elongation Factor-1 (EF-1) gene promoter. An example of an artificial promoting region includes SCP-1 (Super Core Promoter-1). Furthermore, various transcription enhancers may be linked to the promoting regions as disclosed above, such as the transcription enhancing regions derived from murine polyoma and SV40 viruses.

Particularly, animal cells of the present invention are from the type CHO-DG44 of hamster ovary, which lack the enzyme di-hydrofolate reductase (DHFR).

Furthermore, for the selection of cells having their expression vector stably integrated in their genome, various classes of selection markers may be used, being particularly preferred selection markers coding proteins giving resistance against some antibiotics, such as neomicin phosphotransferase and hygromicin phosphotransferase and/or amplifiable selection markers such as the sequences coding enzymes DHFR (dihydrofolate reductase) and glutamine sinthetase.

For the production of endoproteases or light and heavy chains of Factor VIII, the expression vectors do not necessarily need to be stably integrated in the genome of growing cells.

In a particular embodiment, the production of endoproteases may be reached by means of transitory insertion of the expression vector into the nucleus of animal cells. In this case, the production of endoproteases or heavy and light chains of Factor VIII is also transitory.

In another embodiment, the insertion of elements into an expression vector may be conducted, so to allow it, when in the nucleus of animal cells, to be stably replicated and propagated not being integrated to their genome.

Vectors of the present invention may contain various configurations, e. g. one single expression vector containing expression units from both the light chain and one of the used endoproteases, as well as independent vectors for each element to be expressed.

Without any limitation, according to the present invention, the introduction of the expression vector for endoproteases into the growing cells of interest may be made by employing any appropriate method, being particularly preferred: direct absorption, transfection mediated by calcium chloride, transfection mediated by electroporation, lipofection and viral transduction.

Still according to the present invention, endoproteases SPC6, PACE4 AI and PACESOL may be produced in cells CHO-DG44 and later used in the growing medium of cells super-producing Factor VIII to promote proteolytic processing of the light chain or, alternatively, cells producing endoproteases may be used in other configurations to promote proteolytic processing of the light chain of Factor VIII, e. g. in co-cultivation with cells super-producing Factor VIII.

Therefore, with no limitation whatsoever, the present invention includes the proteolytic processing of recombinant human Factor VIII comprising the introduction of coding regions for endoproteases SPC6 and/or PACE4 AI and/or PACESOL in the genome of animal cells producing Factor VIII and/or the addition of endoproteases SPC6 and/or PACE4 AI and/or PACESOL to the growing medium containing recombinant Factor VIII.

In a particular embodiment, endoproteases SPC6, pACE4 AI or PACESOL may be used for the proteolytic processing of other recombinant Factor VIII configurations containing coding regions for light and heavy chains of Factor VIII, interconnected through an artificial connection region or interconnected with paths corresponding to the domain B of varied extensions as disclosed by Lind et al, 1995 (cited above).

The present invention also includes recombinant human Factor VIII, which is obtained by the methods of the present invention, as well as its use to prepare a pharmaceutical product for the treatment of diseases related to the reduction or lack of Factor VIII, like hemophilia A.

The pharmaceutical product of the present invention is formulated with pharmaceutically acceptable excipients as known in the art, for oral or injectable administration.

Pharmaceutically acceptable carriers to make the present invention may include all those known in the art, with no limitation whatsoever. A reference work for the formulation of said pharmaceutical forms is the book Remington's Pharmaceutical Sciences, from the U. S. publisher Mack Publishing.

Excipients are particularly selected from one or more among histidine, Tris, BIS-Tris Propane, PIPES, MOPS, HEPES, MES, ACES, sodium chloride, calcium chloride, calcium gluconate, calcium glubionate, calcium gluceptate, glutathione, homocysteine, trolox, lipoic acid, methionine, sodium thiosulphate, platinum, glycine, BHT, polysorbate, Pluronic polyols, Brij, mannitol, alanine, hydroxyethyl amide, sucrose, trehalose, raffinose, arginine, albumin, etc.

Furthermore, an object of the present invention is to provide methods to treat diseases related to the reduction or lack of Factor VIII, particularly hemophilia A, consisting in the supply to a patient in need of a therapeutically effective quantity of recombinant human Factor VIII which is obtained following the methods of the present invention, particularly in the form of a pharmaceutical product.

The examples below serve to show aspects of the present invention, not having, however, any limitative purpose.

### EXAMPLES

### Enzyme Selection

Six enzymes have been tested, PACE (or furin, abbreviated FUR in the results as presented herein), PACESOL, a new isoform of PACE4 still not characterized (abbreviated as PACE4 in the results as presented herein) having the main domains responsible for PACE4 proteolytic activity, isoform a of PACE4 (abbreviated as PACE4 AI in the results as presented herein), SPC6 and PCSK7, but only SPC6, PACE4 AI and PACESOL show appropriate results, as shown by the examples below.

### Production Method

The method started from growing animal cells producing recombinant human Factor VIII in large quantities. To obtain such cells, coding regions corresponding to the heavy chain of Factor VIII (amino acid residues 1 to 740, preceded by 19 amino acid residues corresponding to the signal peptide of Factor VIII) and the light chain containing a small path corresponding to domain B of Factor VIII (residues 1,563 to 2,332, preceded by 19 amino acid residues corresponding to the signal peptide of Factor VIII) have been stably inserted into the genome of hamster ovary cells CHO-DG44, which lack the enzyme di-hydrofolate reductase (DHFR).

Coding regions as used need to be in a given configuration to be functional in animal cells. Therefore, it is required that coding regions are linked to regulatory elements causing the cell biosynthetic machinery to effectively synthesize the proteic product of interest. For the production of both the heavy chain and the light chain, the following regulatory elements have been used:

a) hybrid promoter region composed by the gene transcription promoter of the β-actin gene of chickens and the transcription enhancer derived from human cytomegalovirus;

b) region of the internal link to the ribosome, known as IRES (Internal Ribosome Entry Site), derived from the encephalomiocardite virus of rabbits. The use of this region allows to link the expression of the coding region of interest to the expression of the selection marker of cells containing the elements of interest integrated to its genome. In our case, the selecion marker as used was the coding region of the enzyme DHFR of mice;

c) A signaling region for the polyadenylation of the transcript or mRNA (messenger RNA) generated from the transcription of the coding region of interest. In our case, the signaling region for polyadenylation derived from the β-globin gene of rabbits has been used.

The coding region of interest, linked to the regulatory elements as disclosed above, constitutes an expression vector of the coding region of interest. Expression vectors of light chain and heavy chain have been co-introduced into CHO-DG44 cells by means of a method known as lypofection.

The following step involved the selection, by means of biochemical complementation, of CHO-DG44 cells stably expressing the enzyme DHFR and consequently containing expression vectors of interest as stably integrated to its genome.

This first cell population, containing expression vectors of interest stably integrated to its genome, has been used to obtain cells by producing Factor VIII in large quantities, by means of a method known as gene co-amplification.

In that method, initially selected cells are submitted to growing concentrations of an inhibitor for the enzyme DHFR, methotrexate (MTX), thus selecting, at the end of each selection cycle, cells producing growing quantities of the enzyme DHFR.

Since the production of the enzyme DHFR is linked to the production of light and heavy chains of Factor VIII, the production of said proteins also increases simultaneously.

Therefore, at the end of the method, CHO-DG44 cells have been obtained, thus producing large quantities of the recombinant human Factor VIII.

Cells producing recombinant Factor VIII as obtained present, as an inconvenience, incomplete proteolytic processing of the light chain.

To induce the proteolytic processing of the light chain, an expression vector for human endoprotease SPC6 (PCSK5), an expression vector for the isoform a of human endoprotease PACE4 (PCSK6) or an expression vector for the mutant as secreted from the human endoprotease PACE, known as PACESOL, has been stably introduced into the genome of CHO-DG44 cells super-producing Factor VIII.

Expression vectors for SPC6, PACE4 AI and PACESOL as used were lentiviral bicistronic expression vectors. Said vectors have elements derived from the HIV (Human Immunodeficiency Virus) virus causing their stable and very effective integration into the genome of target cells. The promoting region of the gene transcription as present in said vector is derived from the human cytomegalovirus.

In that vector, the transcription of the coding region of interest is linked to the transcription of the coding region of a fluorescent protein known as EGFP (Enhanced Green Fluorescent Protein). Said link is provided by the same element IRES as disclosed above.

The fluorescent protein EGFP has been used as an indicator of the stable integration of expression vectors as used in the genome of cells super-producing Factor VIII.

The signaling region for polyadenylation as used was the region known as LTR (Long Terminal Repeat) of the HIV virus. CHO-DG44 cells super-producing Factor VIII thus modified additionally produce recombinant human SPC6 endoprotease or recombinant human PACE4 AI endoprotease or recombinant human PACESOL endoprotease, and started to fully and proteolytically process the light chain of Factor VIII fused to a small path of domain B, making said modified cells to produce just the light chain of Factor VIII identically to what is naturally found in the human plasma, lacking antigene artificial forms.

In another test, the same expression vector for endoprotease SPC6, the same expression vector for endoprotease PACE4 AI and the same expression vector for endoprotease PACESOL have been stably integrated into the genome of CHO-DG44 cells. Said cells started to stably produce and secrete recombinant SPC6, PACE4 AI or PACESOL to the growing medium. Recombinant endoproteases SPC6,

PACE4 AI and PACESOL thus produced have been added as reagents to the growing medium of CHO-DG44 cells super-producing Factor VIII, having also been able to promote, in vitro, the full proteolytic processing of the light chain of Factor VIII.

Assays to Evaluate Proteolytic Processing

FIGS. 5 to 8 are representative of the assay made to evaluate if the proteolytic processing of the light chain of Factor VIII was occurring or not.

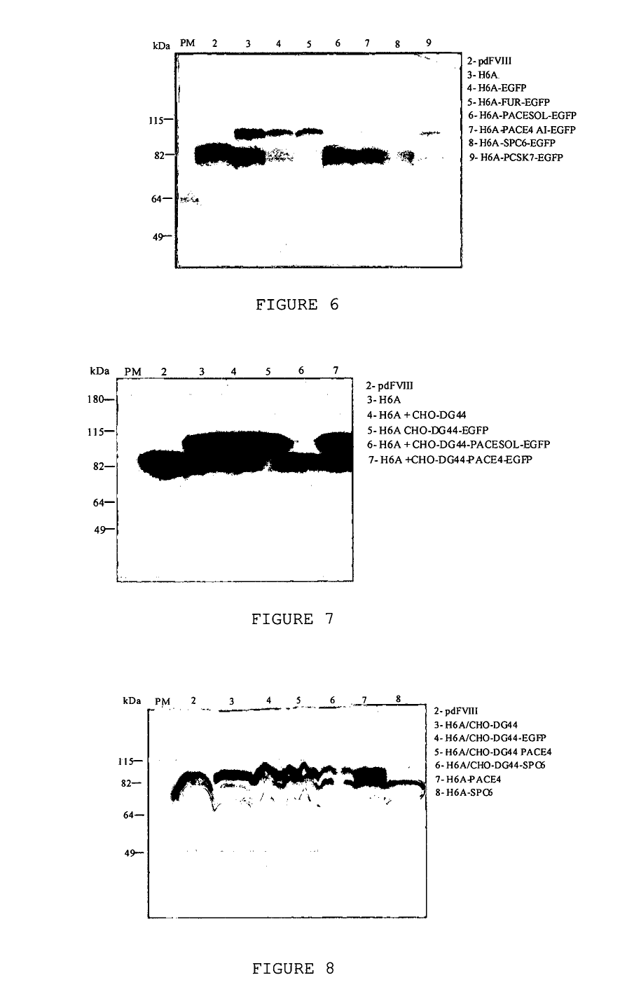

FIGS. 5 and 6 correspond to the assays made to evaluate the alternative of simultaneous production of Factor VIII and proteases.

FIGS. 7 and 8 correspond to the assays to evaluate the alternative to produce proteases in other cells and add them to the growing medium of cells producing Factor VIII. In said assays, PACE (or furin) and PCSK7 enzymes have not been used, due to limitations in their solubilities.

The assay as used is known as Western Blot and, in summary, involves, in a first step, the fractioning of a mixture of proteins, exploring the differences in molecular weight between them and, in a second step, the detection of a given protein by using specific antibodies against it.

In the assay as effected, the protein mixture was provenient from the growing medium as conditioned by the cells producing Factor VIII. Therefore, said growing medium contains, among other components, synthesized proteins secreted by cells producing Factor VIII, which is also secreted to the growing medium. The protein which has been detected in the present assay was the light chain of Factor VIII.

On FIGS. 5 to 8, each numbered vertical band represents an analyzed mixture of proteins. The vertical band indicated as PM corresponds to a mixture of proteins with known molecular masses used as molecular weight standards.

To the left of the figures, the position of said molecular weight markers is indicated (49 kDa and 180 kDa band). Bands 2 at each figure correspond to the positive control for the detection of the light chain of Factor VIII.

As a positive control, a commercial preparation of purified human Factor VIII from donators' plasma has been used. In this control, the detection of a proteic band of approximately 80 kDa, corresponding to the fully processed light chain, is expected.

As one can verify in the results presented in the FIGS. 5 and 6, gutters 3 correspond to the samples of medium conditioned by cells super-producing human Factor VIII, designated as H6A. In those samples, a proteic band has been detected, at the same height of the proteic band as detected in the positive control, corresponding to a fully processed light chain.

A proteic band of about 100 kDa, corresponding to a proteolytically non-processed light chain, has also been detected.

Bands numbered as 5 to 9 on FIGS. 5 and 6 correspond to samples of medium conditioned by genetically modified H6A cells so to produce endoproteases PACE (bands 5, indicated as H6A-FUR-EGFP), the secreted PACE mutant known as PACESOL (bands 6 H6A-PACESOL-EGFP), a new isoform of PACE 4 not yet characterized (band 7 H6A-PACE4-EGFP of FIG. 5), isoform a of PACE 4 (band 8 H6A-PACE4 AI-EGFP of FIG. 6), SPC6 (bands 8 H6A-SPC6-EGFP) and PCSK7 (bands 9 H6A-PCSK7-EGFP). Bands 4 of FIGS. 5 and 6 correspond to the samples of medium as conditioned by genetically modified H6A cells only with the expression vector of endoproteases (H6A-EGFP).

As one can verify on FIGS. 5 and 6, the 100 kDa proteic band corresponding to the non-processed light chain in medium conditioned by H6A-PACESOL-EGFP (bands 6 of FIGS. 5 and 6), H6A-PACE4 AI-EGFP (band 7 of FIGS. 6) and H6A-SPC6-EGFP (bands 8 of FIGS. 6 and 6) cells has disappeared, indicating that the proteolytic processing of the light chain is taking place in those cells.

On FIG. 7, the gutter 3 corresponds to a sample of medium as conditioned by H6A cells super-producing human VIII factor, wherein the same profile as disclosed above has been detected for FIGS. 5 and 6.

Bands with numbers 6 and 7 correspond to samples of medium conditioned by H6A cells growing with medium conditioned by genetically modified parental cells (used to originate H6A cells) CHO-DG44 to produce and secrete endoproteases PACESOL (band 6 H6A+CHO-DG44-PACESOL-EGFP) and a new and not yet characterized isoform PACE 4 (band 7 H6A +CHO-DG44-PACE4-EGFP) to the growing medium.

Band 4 corresponds to the medium conditioned by H6A cells growing with the medium conditioned by cells CHO-DG44 (H6A +CHO-DG44).

Band 5 corresponds to the medium conditioned by H6A cells growing with the medium conditioned by genetically modified CHO-DG44 cells only with the expression vector of endoproteases (H6A+CHO-DG44-EGFP).

As one can observe on FIG. 6 that the 100 kDa proteic band corresponding to non-processed light chain just in the medium conditioned by H6A cells growing with the medium conditioned by CHO-DG44-PACESOL-EGFP cells (band 6) has disappeared, indicating that, upon the addition of PACESOL, in this case produced by another cell line, to the growing medium of cells producing Factor VIII, proteolytic processing of the light chain takes place.

On FIG. 8, the gutter 3 corresponds to a sample of medium as conditioned by H6A cells growing with the medium as conditioned by CHO-DG44 cells, wherein the same profile as disclosed above has been detected for FIGS. 5, 6 and 7.

Bands with numbers 5 and 6 correspond to samples of medium conditioned by H6A cells growing with medium conditioned by genetically modified CHO-DG44 cells to produce and secrete a new and yet not characterized isoform of PACE4 (band 5 H6A +CHO-DG44-PACE4) and endoprotease SPC6 (band 6 H6A +CHO-DG44-SPC6) to the growing medium. Bands 7 and 8 correspond to medium conditioned by genetically modified H6A cells to produce and secrete a new and yet not characterized isoform of PACE4 (band 7 H6A-PACE4) and SPC6 (band 8 H6A-SPC6).

Band 4 corresponds to the medium conditioned by H6A cells growing with the medium conditioned by genetically modified CHO-DG44 cells only with the expression vector of endoproteases (H6A +CHO-DG44-EGFP).

One can observe on FIG. 8 that the 100 kDa proteic band corresponding to non-processed light chain just in the medium conditioned by H6A cells growing with the medium conditioned by CHO-DG44-SPC6-EGFP cells (band 6) has disappeared, indicating that, upon the addition of SPC6, in this case produced by another cell line, to the growing medium of cells producing Factor VIII, proteolytic processing of the light chain takes place. The fact the processing has been partial in this assay is probably due to the use of insufficient quantities of SPC6 to fully perform this processing.

When this endoprotease (SPC6) is produced simultaneously with Factor VIII, processing of the light chain is full (band 8).

It should be realized that the above disclosed embodiments are merely illustrative and any change throughout them may occur for an expert in the art. Consequently, the present invention should not be considered as limited to the embodiments disclosed herewithin.

The expert in the art will know how to promptly evaluate, by means of the teachings as included in the text and in the examples the advantages of the invention and propose variations and equivalent embodiment alternatives, not however escaping from the scope of the invention as defined by the attached claims.

1. Method for the production of recombinant human factor VIII wherein comprises the introduction of coding regions for endoproteases SPC6 and/or PACE4 AI and/or PACESOL into the genome of animal cells producing Factor VIII and/or the addition of endoproteases SPC6 and/or PACE4 AI and/or PACESOL to the growing medium containing recombinant Factor VIII in a method comprising partial deletions of the portion of the coding region for factor VII corresponding to domain B and the independent production of heavy and light chains, in which paths with variable extensions, corresponding to domain B, are connected to the coding region for the light chain.

2. Method according to claim 1 wherein the coding regions for endoproteases SPC6 and/or PACE4 AI and/or PACESOL is stably inserted in the genome of growing animal cells, so that such cells may start to produce endoproteases, to be used, jointly or separately, in the proteolytic processing of artificial forms of Factor VIII, by the addition of endoproteases to the growing medium containing cells producing recombinant human Factor VIII.

3. Method according to claim 1 wherein the production of human endoproteases SPC6, PACE4 AI and PACESOL is effected by means of the translation of their coding region in vitro, by means of chemical synthesis or purification from naturally producing cells.

4. Method according to claim 1 to wherein the endoproteases SPC6, PACE4 Al or PACESOL have amino acid analogues or non-natural amino acids in their sequences, keeping their endoproteolytic activities.

5. Recombinant Human Factor VIII wherein it is obtained according to the method of claim 1.

6.-12. (canceled)
